SEQUENCE LISTING (2 sequences)

SEQ ID NO: 1 - protein, 342 aa, Candida antarctica
MKLLSLTGVAGVLATCVAATPLVKRLPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

SEQ ID NO: 2 - protein, 291 aa, Thermomyces lanuginosus
MRSSLVLFFVSAWTALASPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

(19) United States
(12) Patent Application Publication
(10) Pub. No.: US 2014/0017741 A1
(43) Pub. Date: Jan. 16, 2014

(54) Esterification Process

(71) Applicants: Per Munk Nielsen, Hilleroed (DK); Hans C. Holm, Hellerup (DK)

(72) Inventors: Per Munk Nielsen, Hilleroed (DK); Hans C. Holm, Hellerup (DK)

(73) Assignee: Novozymes A/S, Bagsvaerd (DK)

(21) Appl. No.: 14/004,609

(22) PCT Filed: Mar. 29, 2012
(86) PCT No.: PCT/EP2012/055668

§ 371 Date: Oct. 1, 2013

(30) Foreign Application Priority Data
Mar. 30, 2011 (EP) 11160478.1
Apr. 5, 2011 (EP) 11161192.7

(51) Int. Cl. C12P 7/64

(52) U.S. Cl. CPC C12P 7/649
USPC 435/134

(57) ABSTRACT

The invention relates to the utilisation of fatty acid feedstockin the production of biodiesel by the use of microbial enzymes.

### REFERENCE TO A SEQUENCE LISTING

This application contains a Sequence Listing in computer readable form, which is incorporated herein by reference.

### FIELD OF THE INVENTION

The present invention relates to a novel process for the utilisation of fatty acid feedstock in the production of biodiesel.

### BACKGROUND OF THE INVENTION

As a result of the increasing interest in renewable resources in general and biofuels in particular, a number of processes has been developed for the production of esters of fatty acids and lower, which esters are also referred to as tiodieser. Biodiesel may be produced in a two-step enzymatic process wherein a triglyceride feedstock is reacted with a lower alcohol (e.g., methanol or ethanol) in a first lipase catalysed process step (“transesterification step”) to form fatty acid methyl esters, free fatty acids and free glycerol. The fatty acid methyl esters and free fatty acids are isolated and then reacted with a lower alcohol (e.g., methanol or ethanol) in a second lipase catalysed process step (“esterification step”) to form a fatty acid methyl ester composition with a very low free fatty acid content.

The specifications for biodiesel are becoming stricter with respect to for instance content of metal ions and phospholipids. To meet these specifications distillation of the biodiesel product is required. The distillation is conventionally performed as the last process step after the esterification process. However, further methods for the production of biodiesel are needed.

### SUMMARY OF THE INVENTION

It has surprisingly been found that in the above two-step enzymatic biodiesel process it is advantageous to do the distillation before the esterification process step. The high purity of the feedstock from distillation secures long lifetime of the enzyme in the second lipase catalysed esterification step and facilitates the use of immobilised enzymes, e.g., in a packed bed column. In a conventional biodiesel process the use of immobilised enzymes in a packed bed column would be impractical as the column would clog up due to impurities in the feedstock.

Accordingly, the invention provides a process for the production of fatty acid alkyl esters (FAAE) from a fatty acid feedstock, comprising the steps of: (a) providing a reaction mixture 1 comprising a fatty acid feedstock, a lower alcohol and water; (b) contacting the reaction mixture 1 with a lipase; (c) allowing the reaction mixture 1 to react under formation of fatty acids alkyl esters; (d) separation off the water/glycerol phase from the FAAE/FFA phase of reaction mixture 1; (e) distilling the FAAE/FFA phase of reaction mixture 1 to obtain distilled fatty acid feedstock comprising fatty acids alkyl esters and free fatty acids; (f) providing a reaction mixture 2 that comprises the distilled fatty acid feedstock and a lower alcohol; (g) contacting the reaction mixture 2 with a lipase, and; (h) allowing the reaction mixture 2 to react under formation of fatty acids alkyl esters; wherein water is removed from the reaction mixture 2 during any of the steps (e), (f) and (g).

### DETAILED DESCRIPTION OF THE INVENTION

Biodiesel

Fatty acid alkyl esters (FAAE) of short-chain alcohols, such as fatty acid methyl esters (FAME) and fatty acid ethyl esters (FAEE) are also called biodiesel, because they are used as an additive to or as replacement of fossil diesel.

It is emphasised that the fatty acid alkyl esters produced by the method of the invention is not exclusively for biodiesel, but can also be used as basic oleochemical in further down stream processes in the oleochemical industry.

Alcohol

The alcohol used in the method of the invention is preferably a short-chain, branched or linear, alcohol having 1 to 5 carbon atoms (C₁, C₂, C₃, C₄, or C₅) and mixtures thereof (“a lower alcohol”). Preferred lower alcohols are methanol, ethanol, propanol and mixtures thereof. The alcohol content is preferably less than 4, 3, 2, 1.5 or 1.0 molar equivalents to the amount of fatty acids in the reactant mixture (free and glyceride bound fatty acids). The alcohol may be added stepwise (such as in 1, 2, 3, 4, 5, 6, 7, 8, 9, 10 or more steps) and/or continuously to the reaction mixture over the length of or a part of the reaction period.

Fatty Acid Feedstock

The term “fatty acid feedstock” is defined herein as a fatty acid feedstock substrate comprising triglyceride. The substrate may furthermore comprise fatty acid alkyl esters, diglyceride, monoglyceride, free fatty acid or any combination thereof. Any oils and fats of vegetable or animal origin comprising fatty acids may be used as substrate for producing fatty acid alkyl esters in the process of the invention.

The fatty acid feedstock may be oil selected from the group consisting of: algae oil, canola oil, coconut oil, castor oil, coconut oil (copra oil), corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, and oil from halophytes, pennycress oil, camelina oil, jojoba oil, coriander seed oil, meadowfoam oil, seashore mallow oil, microbial oils or any combination thereof.

The fatty acid feedstock may be fat selected from the group consisting of: animal fat, including tallow from pigs, beef and sheep, lard, chicken fat, fish oil, yellow grease, brown grease, or any combination thereof.

The fatty acid feedstock may be crude, refined, bleached, deodorized, degummed, or any combination thereof.

Food quality oils and fats are expensive, and therefore, waste and by-products from their processing as well as non-food grade oils and fats have become increasingly attractive feedstock for fatty acid alkyl ester. Soap stock is the fraction of oil obtained in an oil refinery by treating the oil with a base to convert free fatty acids to soaps (e.g. sodium soaps). The soap stock usually contains a fraction of glycerides beside the soaps. Acid oil is the by-product from the oil refinery produced by acidification of soap stock to solubilize the soaps. It mainly contains free fatty acids (FFA) and acylglycerols. Distillates like Palm Fatty Acid Distillate (PFAD) is the by-product from oil refining coming from a distillation process used to eliminate free fatty acid from the oil.

The feedstock may be an intermediate product, a waste product or a by-product of oil or fat refining selected from the group consisting of: soap stock; acid oil; fatty acid distillates such as PFAD, soy fatty acid distillate, rapeseed fatty acid distillate, rice bran fatty acid distillate, poultry fat fatty acid distillate, beef tallow fatty acid distillate, etc.; gums from degumming; by-products from the production of omega-3 fatty acids derivates from fish oil; fat trap grease; yellow grease, and brown grease, free fatty acids like oleic acid; or fractions of oil obtained by physical separations; or any combinations thereof.

Enzyme immobilization

The use of immobilized enzymes in processing of oils experience significant growth due to new technology developments that have enabled cost effective methods. A fundamental advantage of immobilized enzymes is that they can be recovered and re-used from a batch process by simple filtration.

Various ways of immobilizing lipases are well known in the art. A review of lipase immobilization is found in “Immobilized lipase reactors for modification of fats and oils a review” Malcata, FX., et al. (1990) J. Am. Oil Chem. Soc. Vol. 67 p. 890-910, where examples of representative lipase immobilizing carriers are illustrated, including inorganic carriers such as diatomaceous earth, silica, porous glass, etc.; various synthetic resins and synthetic resin ion exchangers; and natural polysaccharide carriers such as cellulose and cross-linked dextrin introduced with ion exchange groups.

In some embodiments, the invention relates to a method, wherein the lipase is immobilized either on a carrier; by entrapment in natural or synthetic matrices, such as sol-gels, alginate, and carrageenan; by cross-linking methods such as in cross-linked enzyme crystals (CLEC) and cross-linked enzyme aggregates (CLEA); or by precipitation on salt crystals such as protein-coated micro-crystals (PCMC).

In some embodiments, the invention relates to a method, wherein the carrier is a hydrophilic carrier selected from the group containing: porous inorganic particles composed of alumina, silica or silicates such as porous glas, zeolites, diatomaceous earth, bentonite, vermiculite, hydrotalcite; and porous organic particles composed of carbohydrate polymers such as agarose or cellulose.

In some embodiments, the invention relates to a method, wherein the carrier is a hydrophobic carrier selected from the group containing: synthetic polymers such as nylon, polyethylene, polypropylene, polymethacrylate, or polystyrene; and activated carbon.

Enzyme

A suitable lipase for use in the present invention is a lipase having an enzyme activity classified within E.C. 3.1.1.3. The lipase may have additional activities, such as an activity selected from phospholipase activity, cutinase activity, and acyltransferase activity. The lipase may be a lipase selected from the Candida antarctica lipase A (CALA) as disclosed in WO 88/02775, the C. antarctica lipase B (CALB) as disclosed in WO 88/02775 and shown in SEQ ID NO:1 herein, the Thermomyces lanuginosus (previously Humicola lanuginosus) lipase disclosed in EP 258 068), the Thermomyces lanuginosus variants disclosed in WO 2000/60063 or WO 1995/22615, in particular the lipase shown in positions 1-269 of SEQ ID NO: 2 of WO 95/22615, the Hyphozyma sp. lipase (WO 98/018912), and the Rhizomucor miehei lipase (SEQ ID NO:5 in WO 2004/099400), a lipase from P. alcaligenes or P. pseudoalcaligenes (EP 218 272), P. cepacia (EP 331 376), P. glumae, P. stutzeri (GB 1,372,034), P. fluorescens, Pseudomonas sp. strain SD 705 (WO 95/06720 and WO 96/27002), P. wisconsinensis (WO 96/12012); a Bacillus lipase, e.g., from B. subtilis (Dartois et al. (1993), Biochemica et Biophysica Acta, 1131, 253-360), B. stearothermophilus (JP 64/744992) or B. pumilus (WO 91/16422). Also preferred is a lipase from any of the following organisms: Fusarium oxysporum, Absidia reflexa, Absidia corymbefera, Rhizomucor miehei, Rhizopus delemar (oryzae), Aspergillus niger, Aspergillus tubingensis, Fusarium heterosporum, Aspergillus oryzae, Penicilium camembertii, Aspergillus foetidus, Aspergillus niger, Aspergillus oryzae and Thermomyces lanuginosus, such as a lipase selected from any of SEQ ID NOs: 1 to 15 in WO 2004/099400.

Preferably, the lipase is an enzyme having at least 60%, at least 70%, at least 80%, at least 90%, at least 95%, at least 96%, at least 97%, at least 98%, or even at least 99% identity to any of the aforementioned lipases.

More preferred, the lipase has at least 60%, at least 70%, at least 80%, at least 90%, at least 95%, at least 96%, at least 97%, at least 98%, at least or even at least 99% identity to the amino acid sequence shown as positions 1-342 of SEQ ID NO: 1 herein.

Immobilized lipases suitable for use in the process of the invention and examples of commercially available immobilized lipase include the ones sold under the trade names Novozym 435, Lipozyme RM IM or Lipozyme TL IM, Lipozyme TL 100L, Lipozyme TL HC from Novozymes A/S, Bagsvaerd, Denmark, or Amano PS, from Amano, Japan.

Typically, the enzyme is used in a concentration corresponding to 1 LU/g fatty acid feedstock to 1000 LU/g fatty acid feedstock. Preferably the enzyme is used in a concentration of between 5 LU/g fatty acid feedstock to 500 LU/g fatty acid feedstock, more preferably between 10 LU/g fatty acid feedstock to 100 LU/g fatty acid feedstock.

Typically, the enzyme is used in a concentration corresponding to 1 PLU/g fatty acid feedstock to 1000 PLU/g fatty acid feedstock. Preferably the enzyme is used in a concentration of between 5 PLU/g fatty acid feedstock to 500 PLU/g fatty acid feedstock, more preferably between 10 PLU/g fatty acid feedstock to 100 PLU/g fatty acid feedstock.

Enzyme Sources

The lipase used in the process of the invention may be derived or obtainable from any of the sources mentioned herein. The term “derived” means in this context that the enzyme may have been isolated from an organism where it is present natively, i.e. the identity of the amino acid sequence of the enzyme are identical to a native enzyme. The term “derived” also means that the enzymes may have been produced recombinantly in a host organism, the recombinant produced enzyme having either an identity identical to a native enzyme or having a modified amino acid sequence, e.g. having one or more amino acids which are deleted, inserted and/or substituted, i.e. a recombinantly produced enzyme which is a mutant and/or a fragment of a native amino acid sequence. Within the meaning of a native enzyme are included natural variants. Furthermore, the term “derived” includes enzymes produced synthetically by e.g. peptide synthesis. The term “derived” also encompasses enzymes which have been modified e.g. by glycosylation, phosphorylation etc., whether in vivo or in vitro. The term “obtainable” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by e.g. peptide synthesis. With respect to recombinantly produced enzyme the terms “obtainable” and “derived” refers to the identity of the enzyme and not the identity of the host organism in which it is produced recombinantly.

Accordingly, the lipase may be obtained from a microorganism by use of any suitable technique. For instance, an enzyme preparation may be obtained by fermentation of a suitable microorganism and subsequent isolation of an enzyme preparation from the resulting fermented broth or microorganism by methods known in the art. The enzyme may also be obtained by use of recombinant DNA techniques. Such method normally comprises cultivation of a host cell transformed with a recombinant DNA vector comprising a DNA sequence encoding the enzyme in question and the DNA sequence being operationally linked with an appropriate expression signal such that it is capable of expressing the enzyme in a culture medium under conditions permitting the expression of the enzyme and recovering the enzyme from the culture. The DNA sequence may also be incorporated into the genome of the host cell. The DNA sequence may be of genomic, cDNA or synthetic origin or any combinations of these, and may be isolated or synthesized in accordance with methods known in the art.

The optimum parameters for enzymatic activity will vary depending upon the enzyme used. The rate of enzyme degradation depends upon factors known in the art, including the enzyme concentration, substrate concentration, temperature, the presence or absence of inhibitors and presence of water. These parameters may be adjusted to optimise the esterification reaction.

During an enzymatic treatment step, the temperature of the suspension should be adjusted to provide effective enzyme activity. In general, a temperature of about 30° C. to about 90° C. is used, particularly from about 35° C. to about 60° C.

Process Design

In the present process for enzymatic production of biodiesel a reaction mixture 1 comprising a triglyceride feedstock, a lower alcohol (e.g., methanol or ethanol) and water in a first lipase catalysed transesterification process step forms fatty acid alkyl esters, free fatty acids and free glycerol. Furthermore, glycerol may be added to the reaction mixture 1.

The lipase applied in the first lipase catalysed process step (transesterification step) may be an immobilized lipase on a carrier; the immobilized lipase may be held in suspension in the reaction mixture by agitation. With an immobilized lipase the water content is preferably around 0.5%.

The lipase applied in the first lipase catalysed process step may also be a non-immobilized lipase, i.e., a liquid formulated lipase. In that case the first lipase catalysed process step is preferably performed having a water content in the reaction mixture 1 of from 10% to 50%, from 15% to 40%, or even from 20% to 30%; following the first lipase catalysed process step the reaction mixture 1 the water/glycerol phase containing the non-immobilized lipase is separated from the reaction mixture 1 and may be recycled to new batches of triglyceride feedstock. The oil phase of the reaction mixture 1 is isolated by distillation to yield a very pure fatty acid alkyl ester and free fatty acid fraction having a water content of no more than 500 ppm of water, 300 ppm of water, or even 200 ppm of water. The term distillation is intended to include any type of distillation process such as steam distillation, vacuum stripping and deodorization (Lipid Handbooks 245. 3ed ed. Gunstone et al. CRC Press 2007). A reaction mixture 2 is formed by the fatty acid alkyl ester and free fatty acid fraction and a lower alcohol (e.g., methanol or ethanol). In the second lipase catalysed process step (esterification step) the remaining free fatty acids react with the lower alcohol and a fatty acid alkyl ester fraction with very low FFA content is formed. The second lipase catalysed process step is preferably performed using immobilised enzymes, e.g., by passing reaction mixture 2 through the immobilised enzymes in a packed bed column. Water is a reaction product and needs to be removed from the reaction mixture 2 throughout the reaction period to drive the process to the ester-side—either in a number of steps (e.g. by vacuum treatment as in Example 1) or continuously (e.g. by stripping with an inert gas flow such as in US2010/167360). The final product after the second lipase catalysed process step is a very pure fatty acid alkyl ester fraction having less than 0.25% FFA, less than 0.20% FFA, less than 0.15% FFA or even less than 0.10% FFA and/or less than 300 ppm water, less than 200 ppm water, less than 100 ppm water, or even less than 50 ppm water. The lower alcohol applied in the first lipase catalysed process step for transesterification of the triglyceride may be any lower alcohol, preferably methanol, ethanol and/or propanol. The lower alcohol applied in the second lipase catalysed process step for esterification of the FFA may be any lower alcohol, preferably methanol, ethanol and/or propanol. Different lower alcohols may be applied in the first and the second lipase catalysed process steps (i.e. in the reaction mixtures 1 and 2). In a preferred embodiment ethanol is applied in the first lipase catalysed process step and methanol is applied in the second lipase catalysed process step.

Materials and Methods

Degree of identity

For purposes of the present invention, the degree of identity may be suitably determined according to the method described in Needleman, S. B. and Wunsch, C. D., (1970), Journal of Molecular Biology, 48, 443-45, with the following settings for polypeptide sequence comparison: GAP creation penalty of 3.0 and GAP extension penalty of 0.1. The determination may be done by means of a computer program known such as GAP provided in the GCG program package (Program Manual for the Wisconsin Package, Version 8, August 1994, Genetics Computer Group, 575 Science Drive, Madison, Wis., USA 53711).

Two given sequences can be aligned according to the method described in Needleman and Wunsch (supra) using the same parameters. This may be done by means of the GAP program (supra).

Lipolytic Activity

The lipolytic activity may be determined using tributyrin as substrate. This method is based on the hydrolysis of tributyrin by the enzyme, and the alkali consumption to keep pH constant during hydrolysis is registered as a function of time. One Lipase Unit (LU) is defined as the amount of enzyme which, under standard conditions (i.e. at 30° C.; pH 7.0; with 0.1% w/v Gum Arabic as emulsifier and 0.16 M tributyrin as substrate) liberates 1 micromol titrable butyric acid per minute. One KLU is 1000 LU.

The ester synthesis activity of immobilised lipases may be determined as Propyl Laurate Units per gram product: PLU/g. The immobilised lipase esterify lauric acid with 1-propanol, forming propyl laurate. The activity (μmol/g/min) is determined by quantification of formed propyl laurate and consumed lauric acid by GC.

One PLU unit is defined as the amount of enzyme which, under standard conditions (i.e. at 60° C. and reaction time 20 min.) forms 1 micromole propyl laurate per minute.

Enzymes

Novozym 435 is a commercial enzyme product from Novozymes A/S comprising an immobilized lipase B from Candida antarctica (SEQ ID NO: 1 herein). The product has an activity of 10000 PLU/g.

Lipex 100 L is a commercial enzyme product from Novozymes A/S comprising an immobilized lipase from Thermomyces lanuginosus (SEQ ID NO: 2 herein). The product has an activity of 100 KLU/g.

Lipozyme TL 100L is a commercial enzyme product from Novozymes A/S comprising a liquid formulated lipase from Thermomyces lanuginosus (SEQ ID NO: 2 herein). The product has an activity of 100 KLU/g.

### EXAMPLE 1

A process of the invention may comprise:
1. transesterification with
   a. liquid formulated lipase, or

b. immobilized lipase, 2. separation of the FAME/FFA ester phase from water/glycerol phase, 3. distillation off of the FAME and FFA from the FAME/FFA phase, and 4. esterification of the FFA in the distillate.

1 a.

Soybean oil is mixed with water and glycerol in the ratio 80:10:10 w/w and adjusted to 40° C. Liquid formulated enzyme (Lipozyme TL 100L, Novozymes Denmark) is added at dosage 0.5% of the oil. The reaction mixture is incubated with stirring at 40° C. for 22 hours. Methanol is added in an amount of 1.3 molar equivalents relative to the fatty acids in the oil (14.1 g methanol to 100 g oil). The methanol is added gradually over the first 5 hours of incubation. When the reaction is finalized the content of FAME and FFA in the FAME/FFA phase is expected to be approximately 92% and 6%, respectively.

1 b.

Soybean oil is mixed with water and glycerol in the ratio 80:10:10 w/w and adjusted to 40° C. Immobilized enzyme (Lipozyme TL HC, Novozymes Denmark) is added at dosage 5% of the oil.

The reaction mixture is incubated with stirring at 40° C. for 22 hours. Methanol is added in an amount of 1.3 molar equivalents relative to the fatty acids in the oil (14.1 g methanol to 100 g oil). The methanol is added gradually over the first 5 hours of incubation. When the reaction is finalized, the expected content of FAME and FFA in the FAME/FFA phase is expected to be approximately 92% and 6%, respectively.

2.

The FAME/FFA phase is obtained by decanting off the water/glycerol phase. When liquid enzyme is used the water/glycerol phase contains the enzyme and can be re-used for further batches of FAME production.

3.

The FAME/FFA phase is distilled to obtain distilled fatty acid feedstock expected to be composed of 6% oleic acid and 94% FAME.

4.

The distilled fatty acid feedstock is (a) dried by vacuum treatment at 50° C. to a water content of <200 ppm, (b) four % methanol is added and (c) this reaction mixture is pumped through a packed bed column with immobilized lipase (Novozym 435, Novozymes Denmark) held at 40° C. This treatment (a, b and c) is performed three times and the final reaction product is vacuum dried to a water content <200 pm. By the three passes through the column the FFA content is expected to be reduced to <0.25% and the product is ready for use as biodiesel. The wet methanol collected from the vacuum drying of the reaction mixture can be re-used in the transesterification in step 1(a or b) as this reaction is tolerant to water.

1. A process for the production of fatty acid alkyl esters (FAAE) from a fatty acid feedstock, comprising the steps of:
(a) providing a reaction mixture 1 comprising a fatty acid feedstock, a lower alcohol and water;
(b) contacting the reaction mixture 1 with a lipase;
(c) allowing the reaction mixture 1 to react under formation of fatty acids alkyl esters;
(d) separation off the water/glycerol phase from the FAAE /FFA phase of reaction mixture 1;
(e) distilling the FAAE/FFA phase of reaction mixture 1 to obtain distilled fatty acid feedstock comprising fatty acids alkyl esters and free fatty acids;
(f) providing a reaction mixture 2 that comprises the distilled fatty acid feedstock and a lower alcohol;
(g) contacting the reaction mixture 2 with a lipase; and
(h) allowing the reaction mixture 2 to react under formation of fatty acids alkyl esters;
wherein water is removed from the reaction mixture 2 during any of the steps (e), (f) and (g).

2. The process of claim 1, wherein steps (e), (f) and (g) are repeated one or more times.

3. The process according to claim 1, wherein water is removed from reaction mixture 2 during any of the steps (f), (g) and (h) by stripping with an inert gas, by flashing, or continuous evaporation.

4. The process according to claim 1, wherein the amount of water during any of the steps (f), (g) and (h) are maintained below 300 ppm.

5. The process according to claim 1, wherein the fatty acid feedstock is derived from any of the group consisting of: algae oil, canola oil, coconut oil, castor oil, coconut oil (copra oil), corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, and oil from halophytes, pennycress oil, camelina oil, jojoba oil, coriander seed oil, meadowfoam oil, seashore mallow oil, microbial oils or any combination thereof.

6. The process according to claim 1, wherein the alcohol(s) in step (a) and/or step (f) is selected from methanol, ethanol, and propanol, including mixtures thereof.

7. The process according to claim 1, wherein the lipase in step (b) is a liquid formulated lipase and/or an immobilized lipase.

8. The process according to claim 1, wherein the lipase in step (b) is a liquid formulated lipase and the water content in the reaction mixture 1 is from 10% to 50%.

9. The process according to claim 1, wherein step (f) is performed using an immobilized lipase.

10. The process according to claim 1, wherein the lipase is immobilized on a carrier, such as a hydrophilic carrier selected from the group containing: porous inorganic particles composed of alumina, silica or silicates such as porous glas, zeolites, diatomaceous earth, bentonite, vermiculite, hydrotalcite; and porous organic particles composed of carbohydrate polymers such as agarose or cellulose, or such as a hydrophobic carrier selected from the group containing: synthetic polymers such as nylon, polyethylene, polypropylene, polymethacrylate, or polystyrene; and activated carbon.

11. The process according to claim 1, wherein step (g) is performed in a packed bed column.

12. The process according to claim 1, wherein the lipase in step (e) and/or (f) is the Candida antarctica lipase B and/or an enzyme having at least 60%, at least 70%, at least 80%, at least 90%, at least 95%, at least 96%, at least 97%, at least 98%, at least or even at least 99% identity to the amino acid sequence shown as SEQ ID NO: 1.

13. The process according to claim 1, wherein the lipase in step (e) and/or (f) is the Thermomyces lanuginosus lipase and/or an enzyme having at least 60%, at least 70%, at least 80%, at least 90%, at least 95%, at least 96%, at least 97%, at least 98%, at least or even at least 99% identity to the amino acid sequence shown as SEQ ID NO: 2.

14. The process according to claim 1, wherein the water/glycerol phase from reaction mixture 1 containing the enzyme is reused in a transesterification reaction.

15. The process according to claim 1, wherein the lower alcohol applied in reaction mixture 1 is ethanol and the lower alcohol applied in reaction mixture 2 is methanol.
